(19) United States
(12) Patent Application Publication
(10) Pub. No.: US 2026/0202304 A1
(43) Pub. Date: Jul. 16, 2026

(54) FLOW CYTOMETRY WAVEFORM PROCESSING

(71) Applicant: Beckman Coulter, Inc., Brea, CA (US)

(72) Inventors: Michael KAPINSKY, Bavaria (DE); Larry R. MYERS, Greenfield, IN (US); Robert J. ZIGON, Carmel, IN (US)

(73) Assignee: Beckman Coulter, Inc., Brea, CA (US)

(21) Appl. No.: 19/112,877

(22) PCT Filed: Sep. 26, 2023
(86) PCT No.: PCT/US2023/033685

§ 371 Date: Mar. 18, 2025

Related U.S. Application Data
(60) Provisional application No. 63/410,984, filed on Sep. 28, 2022.
(60) Provisional application No. 63/483,396, filed on Feb. 6, 2023.

(51) Int. Cl. G01N 15/1429; G01N 15/14; G01N 15/1434

(52) U.S. Cl. CPC G01N 15/1429; G01N 15/1434; G01N 15/1459; G01N 2015/1402

(57) ABSTRACT

Systems and methods for analyzing flow cytometry particles. A flow cytometry system is configured to direct a fluid stream of particles through an interrogation location, and includes a laser configured to emit light toward the interrogation location to produce light signals from the particles, and one or more detectors configured convert the light signals to waveform data. The flow cytometry system includes a waveform acquisition device to continuously digitize the waveform data, and a graphics processing unit configured to apply one or more adjustable threshold voltages to the digitized waveform data to extract event data for the particles.

This application is being filed on Sep. 26, 2023, as a PCT International Patent application and claims the benefit of and priority to U.S. provisional patent application No. 63/410,984, filed Sep. 28, 2022, and U.S. provisional patent application No. 63/483,396, filed Feb. 6, 2023, the entire disclosures of which are incorporated herein by reference in their entireties.

### BACKGROUND

Flow cytometry is a technique for detecting and analyzing chemical and physical characteristics of cells or particles in a fluid sample. A flow cytometer may be used to assess cells from blood, bone marrow, tumors, and other body fluids. Typically, the sample is passed through a fluid nozzle which aligns particles in a single file line within a sheath fluid. A laser beam illuminates the particles as the particles pass through in single file to generate radiated light including forward scattered light, side scattered light, and fluorescent light. The radiated light can then be detected and analyzed to determine one or more characteristics of the particles.

### SUMMARY

In general terms, the present disclosure relates to analyzing particles using flow cytometry. In one possible configuration, one or more adjustable threshold voltages are applied to digitized waveform data to extract event data from particles passing through an interrogation location of a flow cytometer without re-running the particles through the interrogation location.

One aspect relates to a flow cytometry system configured to direct a fluid stream of particles through an interrogation location, the flow cytometry system comprising: a laser configured to emit light toward the interrogation location to produce light signals from the particles; one or more detectors configured to convert the light signals to waveform data; a waveform acquisition device configured to digitize the waveform data; and a graphics processing unit configured to apply one or more adjustable threshold voltages to the digitized waveform data to extract event data from the particles.

Another aspect relates to a method of analyzing particles flowing through a flow cytometer, the method comprising: directing a fluid stream of particles through an interrogation location; emitting light toward the interrogation location to produce light signals from the particles; converting the light signals to analog waveform data; continuously digitizing the analog waveform data including time between the particles when a particle is not interrogated by the laser; and applying one or more adjustable threshold voltages to the digitized waveform data to extract event data from the particles.

Another aspect relates to a non-transitory computer readable medium embodying program instructions, which when executed by a processor, cause the processor to: digitize voltage waveforms generated as particles flow through an interrogation location; store the digitized voltage waveforms; apply first and second thresholds to the digitized voltage waveforms; and generate event data including a sequence of digital values from the digitized voltage waveforms by having each digital value in the sequence of digital values be greater than the first threshold and less than the second threshold without re-running the particles through the interrogation location.

Another aspect relates to a method of operating a particle analyzer, the method comprising: passing particles through an interrogation location; irradiating the particles with light as the particles pass through the interrogation location; detecting light signals from the particles; generating digitized waveform data from the light signals; and storing the digitized waveform data in persistent storage.

Another aspect relates to a method of post-processing flow cytometry data, the method comprising: accessing digitized waveform data from a computer readable storage device after interrogation of particles in a sample has been completed by a flow cytometer; determining a threshold voltage; applying the threshold voltage to the digitized waveform data; and characterizing the particles in the sample after applying the threshold voltage.

Another aspect relates to a flow cytometry system configured to direct a fluid stream of particles through an interrogation location, the flow cytometry system comprising: a laser configured to emit light toward the interrogation location to produce light signals from the particles; one or more detectors configured convert the light signals to waveform data; a waveform acquisition device configured to digitize the waveform data; a graphics processing unit configured to apply one or more adjustable threshold voltages to the digitized waveform data to extract event data from the particles; and a graphical user interface to display the one or more adjustable threshold voltages relative to the digitized waveform data; wherein in response to a change of the one or more adjustable threshold voltages, the graphics processing unit is configured to generate updated event data based on the change of the one or more adjustable threshold voltages without re-running the particles through the interrogation location.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### BRIEF DESCRIPTION OF THE DRAWINGS

The following drawing figures, which form a part of this application, are illustrative of the described technology and are not meant to limit the scope of the disclosure in any manner.

FIG. 1 is a schematic block diagram illustrating an example of a flow cytometer system.

FIG. 2A shows a particle entering a laser beam at an interrogation location in the flow cytometer system of FIG. 1.

FIG. 2B shows the particle passing through a center area of the laser beam at the interrogation location in the flow cytometer system of FIG. 1.

FIG. 2C shows the particle exiting the laser beam at the interrogation location in the flow cytometer system of FIG. 1.

FIG. 3 illustrates an example of waveform data plotted with respect to a threshold value that can be generated by the flow cytometer system of FIG. 1.

FIG. 4 is a block diagram of a waveform analysis device of the flow cytometer system of FIG. 1.

FIG. 5 illustrates an example graphical user interface (GUI) that can be generated by the waveform analysis device of FIG. 4.

FIG. 6 shows an example waveform display window that can be generated by the waveform analysis device of FIG. 4, the waveform display window graphically displaying unthresholded waveform data.

FIG. 7 shows an example of a high threshold value applied to the waveform data in the waveform display window of FIG. 6.

FIG. 8 shows an example of an intermediate threshold value applied to the waveform data in the waveform display window of FIG. 6.

FIG. 9 shows an example of a low threshold value applied to the waveform data in the waveform display window of FIG. 6.

FIG. 10 shows an example of multiple thresholds applied to the waveform data in the waveform display window of FIG. 6.

FIG. 11 shows an example of a non-linear threshold applied to the waveform data in the waveform display window of FIG. 6.

FIG. 12 shows an example of event-specific thresholds applied to the waveform data in the waveform display window of FIG. 6.

FIG. 13 is a block diagram of another example of the flow cytometer system of FIG. 1.

FIG. 14 shows an example of a waveform display window graphically displaying waveform plots for data collected by the flow cytometer system of FIG. 13.

FIG. 15 is a flowchart illustrating an example of a method of analyzing particles in the flow cytometry system of FIG. 1.

FIG. 16 is a flowchart illustrating another example of a method of analyzing particles in the flow cytometry system of FIG. 1.

FIG. 17 illustrates an exemplary architecture of a computing device that can be used to implement aspects of the flow cytometry system of FIG. 1.

FIG. 18 illustrates another example of a graphical user interface (GUI) that can be generated by the waveform analysis device of FIG. 4.

FIG. 19 schematically illustrates another example of the flow cytometer system of FIG. 1, the flow cytometry system including a host random-access memory (RAM).

FIG. 20 schematically illustrates an example of a method of updating the GUI of FIG. 18 based on adjustments of an upper or lower threshold slider.

FIG. 21 schematically illustrates an example of a method of decimating waveform data that can be performed by the waveform analysis device of FIG. 4.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

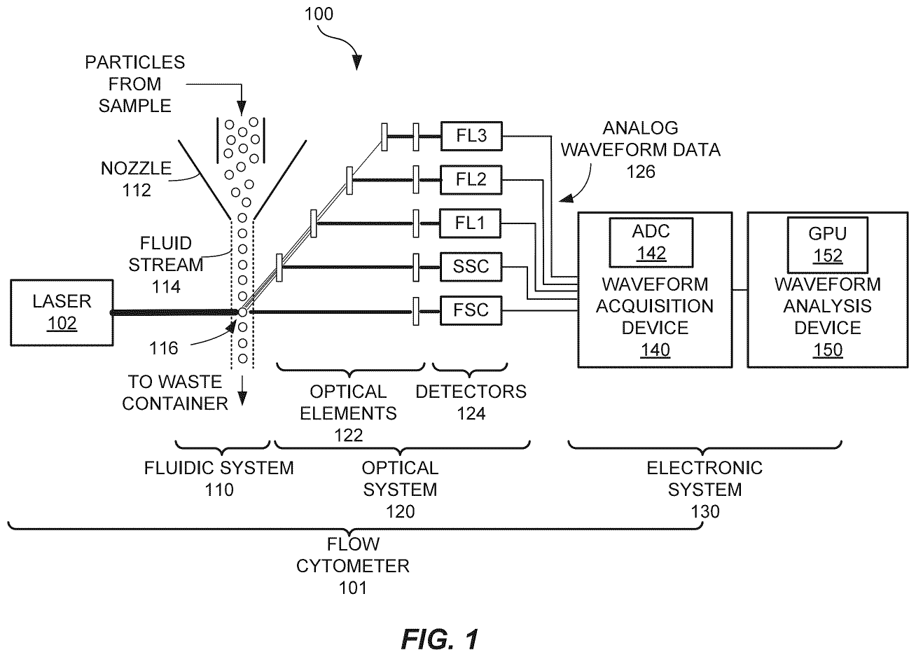

FIG. 1 is a schematic block diagram illustrating an example of a flow cytometer system 100. In general, the flow cytometer system 100 can be used to measure and analyze physical and chemical properties of a sample of particles or cells. For example, the flow cytometer system 100 can collect data from millions of cells in a matter of minutes for display in a variety of formats for researchers or clinicians. Some example applications implemented on the flow cytometer system 100 can include phenotyping to identify and count specific cell types within a population, analyzing DNA or RNA content, determining the presence of antigens on the surface or within cells, and assessing cell health status.

The flow cytometer system 100 generally includes three main component subsystems: a fluidic system 110, an optical system 120, and an electronic system 130. The fluidic system 110 includes a nozzle 112 which receives a sample containing particles or cells suspended in a fluid. The nozzle 112 creates and ejects a fluid stream 114 of particles arranged in a single file line. Each particle passes through one or more beams of light produced by a laser 102. The point at which a particle intersects with a light beam is known as an interrogation location 116.

The optical system 120 includes the laser 102, optical elements 122, and detectors 124. At the interrogation location 116, light from the laser 102 hits a particle and scatters. The optical elements 122 direct the scattered light toward the detectors 124. The detectors 124 may include a forward scatter (FSC) detector to measure scatter along the path of the laser 102, a side scatter (SSC) detector to measure scatter at a ninety-degree angle relative to the laser 102, and/or one or more fluorescence detectors (e.g., FL1, FL2, and FL3) to measure the emitted fluorescence intensity of different wavelengths of light.

Generally, FSC intensity is proportional to the size or diameter of the particle due to light diffraction around the particle. FSC may therefore be used for the discrimination of particles by size. SSC, on the other hand, is produced from light refracted or reflected by internal structures of the particle and may therefore provide information about the internal complexity or granularity of the particle. By adding fluorescent labelling to a sample, different fluorescent signals/channels (e.g., green, orange, and red) can be analyzed for functional characteristics of a cell. For example, since T-cells present CD3 binding sites, a sample containing T-cells may be “stained” with anti-CD3 antibodies conjugated with a fluorescent molecule. As these cells pass through the interrogation location 116, the laser light excites the fluorescent tag, or fluorochrome, to emit photons at a wavelength detectable by a fluorescence detector. The detectors 124 may therefore simultaneously measure a number of parameters and enable categorization of particles by their function based on detected wavelengths of light.

The electronic system 130 includes a waveform acquisition device 140 and a waveform analysis device 150. The waveform acquisition device 140 is communicatively coupled with the detectors 124 and is configured to receive analog waveform data 126 generated by the detectors 124. The waveform acquisition device 140 includes an analog-to-digital converter (ADC) 142 configured to digitize the waveform data.

The waveform analysis device 150 is configured to receive the digital waveform data and display it for a user of the flow cytometer system 100. In some embodiments, the waveform analysis device 150 comprises a computing device communicatively coupled with a flow cytometer 101 over a network, and the flow cytometer 101 may include the fluidic system 110, optical system 120, and the waveform acquisition device 140. In other embodiments, the waveform analysis device 150 is integrated with the flow cytometer 101.

Current flow cytometers use a field-programmable gate array (FPGA) in the waveform acquisition device 140 to obtain information about individual particles passing through the interrogation location. In current flow cytometers, the waveform acquisition device 140 uses a single threshold value to determine when the output of the detectors begins conversion from analog to digital. Only a single threshold value can be used for a single run of a sample through the current flow cytometer. The threshold value is a constant value and may be referred to as a voltage threshold value. As such, if or when a detector outputs a voltage value that crosses the threshold, digitization begins and the digital value is sent to the FPGA. As waveform data is digitized, the FPGA computes the height, width, and area of each pulse. Other data relating to the waveform, including data not exceeding the voltage threshold value, is not captured, stored, or otherwise available for analysis. Additionally, if a user wishes to adjust the threshold value, the sample must be collected from the waste container and the experiment re-run with the new threshold value, incurring costs in resources and time.

To address the above issues, the flow cytometer system 100 is enhanced with a graphics processing unit (GPU) 152 as a component of the waveform analysis device 150. The GPU 152 is configured to process a continuous digital stream generated by the waveform acquisition device 140 and provided to the waveform analysis device 150. The digital stream is continuous in that the waveform acquisition device 140 does not threshold the waveform data produced by the detectors 124. The FPGA may also be removed or excluded from the waveform acquisition device 140. Instead, during an experiment, the waveform acquisition device 140 continuously digitizes the analog waveform data 126 at a high rate (e.g., 1 GHz) without thresholding. As an illustrative example, the waveform acquisition device 140 can have a sampling rate of about 1 GHz, which can result in digital waveform files that are approximately 1,000 times larger than those typically generated by the FPGA in current flow cytometers to produce the area, height, and width values. In some examples, the waveform acquisition device 140 is configured to continuously digitize the analog waveform data including time between the particles when a particle is not interrogated by the laser in the interrogation location 116.

The waveform analysis device 150 may thus receive a digitized version of the waveform data with increased data points, and the waveform data for an experiment is unthresholded and available in its entirety for processing by the GPU 152. In addition to having the capability of processing a large stream or file of waveform data, the GPU 152 enables thresholding the waveform at the post-processing step as opposed to the waveform acquisition step. This in turn provides several technical benefits including the ability to dynamically adjust thresholds and update graphical plots in real-time without re-running an experiment. The GPU 152 may also measure and extract biologically relevant information present the waveform data beyond the three parameters of height, width, and area. Further details of operation and advantages are discussed below.

The flow cytometer system 100 shown in FIG. 1 includes elements which are shown and described for purposes of discussion and it will be appreciated that numerous variations in components and functions are possible. The optical elements 122 may include a series of filters, dichroic mirrors, and/or beam splitters to select out different wavelengths of light and provide the wavelength to an appropriate detector. The detectors 124 may include, for example, photomultiplier tubes (PMTs) or avalanche photodiodes (APDs).

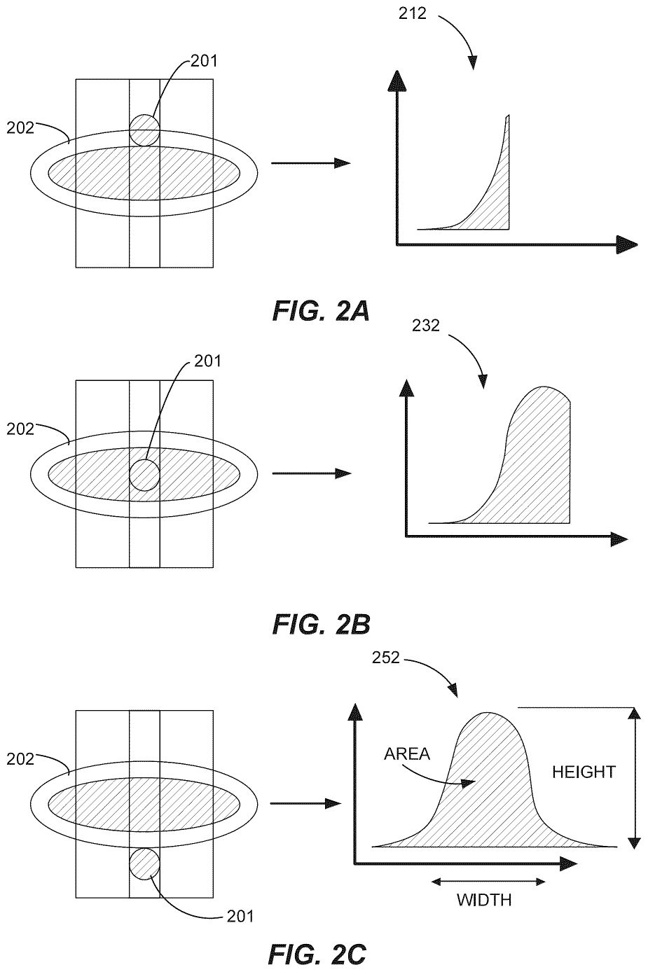

FIGS. 2A-2C illustrate waveform data generated by the detection of a particle 201 passing through a laser beam 202 at the interrogation location 116 in the flow cytometer system 100. As the particle 201 passes through the interrogation location 116 of the light source, a pulse is generated in one or more of the detectors 124. FIG. 2A shows the particle 201 entering the laser beam 202. As the particle 201 starts to intersect with the laser beam 202 it begins to generate scattered light and fluorescence signals. The detector 124 produces a current or voltage that is proportional to the number of photons that hit the photocathode. As such, the output of the detector 124 begins to rise as shown in plot 212 due to current flowing in the detector 124.

FIG. 2B shows the particle 201 passing through a center area of the laser beam 202 at the interrogation location 116. As the particle 201 continues to move downward into a center of the laser beam 202 the particle 201 is fully illuminated. Since photon density of the laser beam 202 is highest in the center a maximum amount of optical signal is produced. The current or voltage of the detector 124 therefore peaks as shown in plot 232.

FIG. 2C shows the particle 201 exiting the laser beam 202 at the interrogation location 116. As the particle flows out of the laser beam 202, the current or voltage output of the detector 124 returns to baseline, as shown in plot 252. This generation of a pulse is called an event. The height is the maximum current/voltage output by the detector 124, the width is the time interval during which the pulse occurs, and the area is the integral of the pulse. Generally speaking, the height and area correspond with signal intensity and the width corresponds with the time the particle is illuminated by the laser beam 202. Accordingly, as pulses are generated, the pulses may be quantified by height, width, and area. This information may be used to distinguish between particles and fluorescence signals may be displayed on plots, analyzed, and interpreted.

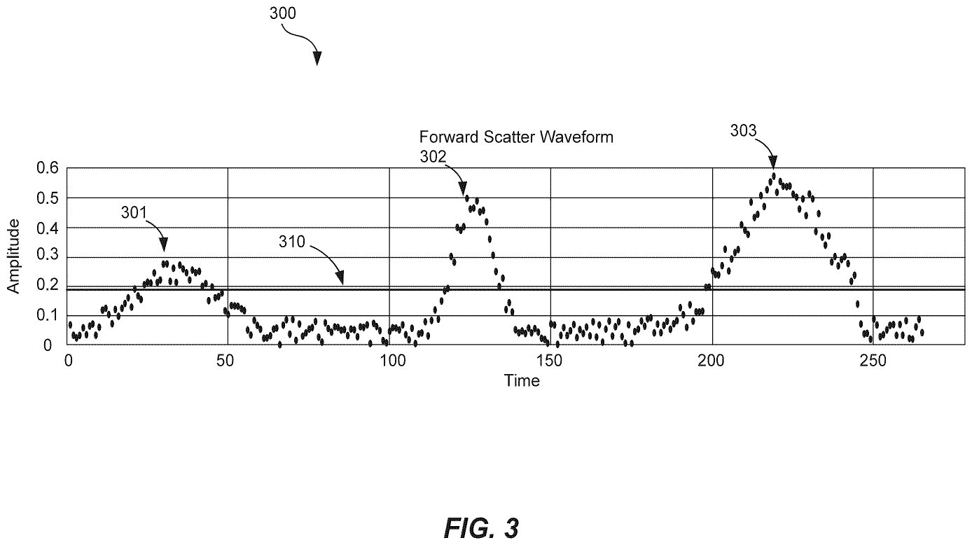

FIG. 3 illustrates an example of waveform data 300 plotted with respect to a threshold value 310 that can be generated by the flow cytometer system 100. In this example, the threshold value 310 is a single constant threshold voltage. As described above, in traditional polychromatic and spectral flow cytometry, the threshold value 310 is used to specify when the digitization of detector output (e.g., analog waveform data 126) begins. That is, when the waveform data 300 travels above the threshold value 310, the waveform acquisition device begins computing the height, width, and area of each pulse 301-303 that is above the threshold value 310. Waveform data 300 that is below the threshold value 310 is discarded in prior techniques.

The problem with the above-described approach is that the threshold value 310 may not be appropriately set for the entire voltage waveform for the purpose of extracting event data. For instance, the threshold value 310 of this example may be set too high to accurately analyze cells generating a pulse similar to the pulse 301 of the waveform data 300. On the other hand, if the threshold value 310 is set too low it may compromise the overall signal-to-noise ratio of the waveform data 300. Additionally, in conventional flow cytometers, the single threshold value must be set prior to data acquisition, irreversibly discarding events of potential relevance.

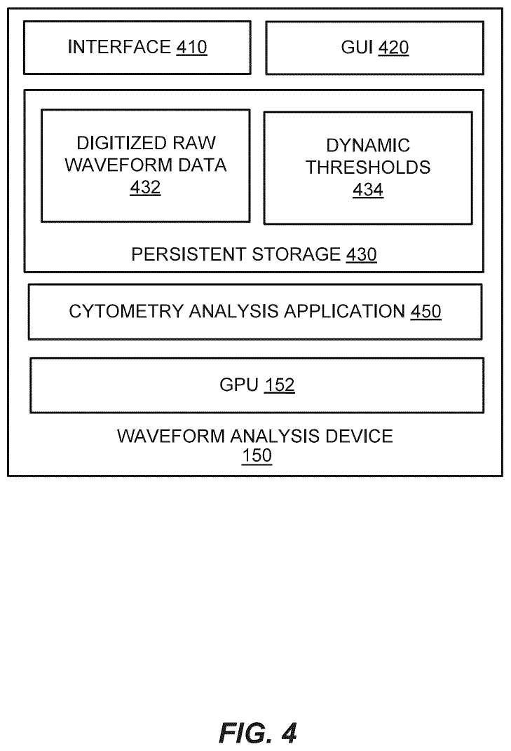

FIG. 4 is a block diagram of an example of the waveform analysis device 150. The waveform analysis device 150 can receive, store, and display waveform data that has been continuously sampled without having been thresholded upstream at the waveform acquisition device 140. The waveform analysis device 150 can include an interface 410 to receive digitized raw waveform data 432, persistent storage 430 to store the digitized raw waveform data 432, and a graphical user interface (GUI) 420 to display the digitized raw waveform data 432. The persistent storage 430 may also store a plurality of dynamic thresholds 434 that allow for non-linear thresholding and real-time updating and displaying of applied thresholds as further described below. The persistent storage 430 may comprise system memory such as random access memory (RAM) and/or long term non-volatile memory such as a hard drive.

The waveform analysis device 150 may further include a cytometry analysis application 450 comprising a software application or a set of related software applications configured to instruct the GPU 152 to process the digitized raw waveform data 432. The cytometry analysis application 450 may execute on one or more processors (not shown) to provide other functions described herein in conjunction with the GPU 152 such as receiving user input via the GUI 420. One or more components of the waveform analysis device 150 may reside in a cloud computing application in a network distributed system. In that regard, the waveform analysis device 150 may be any of a variety of computing devices, including, but not limited to, a personal computing device, a server computing device, or a distributed computing device.

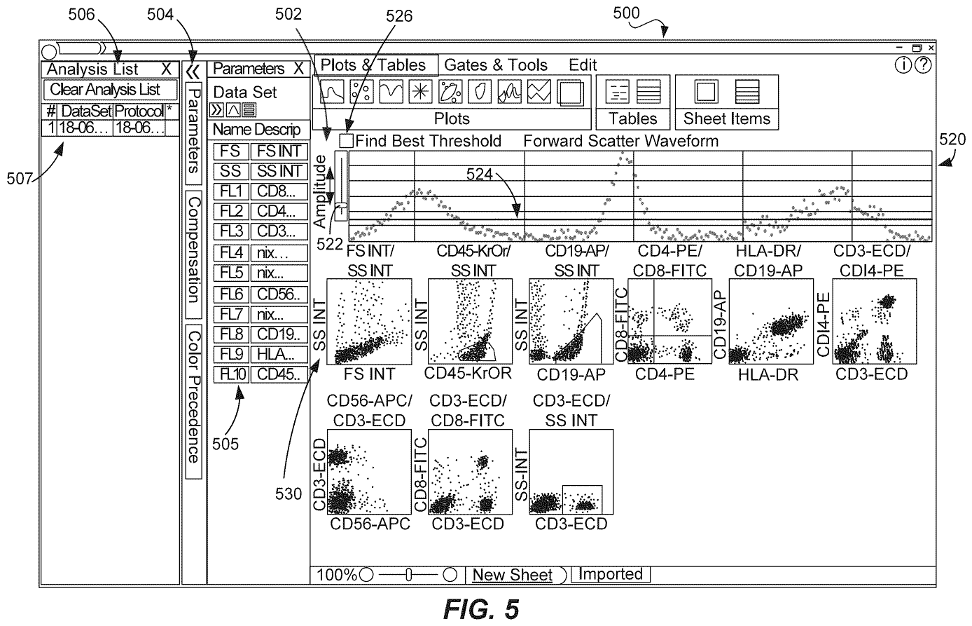

FIG. 5 illustrates an example graphical user interface (GUI) 500 that can be generated by the waveform analysis device 150. The GUI 500 includes a waveform display window 502 to display graphs and plots of waveform data, a parameters window 504 for selecting parameters 505 to display in the waveform display window 501, and a data set window 506 to select a file or data set 507 to be processed and displayed. A user may select a data set 507 stored in persistent storage 430 of the waveform analysis device 150, and select the parameters 505 to display for the data set 507. A parameter in this context is a measurement from a particular detector 124 of the flow cytometer system 100. The parameters may be used to generate graphs and plots including waveform graphs, histograms, scatter plots, density plots, comparison plots, and the like. In this example, the waveform display window 502 displays a forward scatter waveform 520 and a plurality of scatter plots 530 related to side scatter and fluorescence intensity.

The GUI 500 includes an adjustable threshold element 522 that is selectable by a user to adjust the threshold 524 to a higher or lower value. For example, the adjustable threshold element 522 can be moved or dragged along a scale, as indicated by the double arrow, to adjust the threshold 524. Each time the threshold 524 is reset or updated in the GUI 500, the GPU 152 applies the new threshold value(s) to the waveform data. The GPU 152 extracts measurements according to the new threshold value(s) and updates each of the graphs and plots displayed in the waveform display window 501 in real-time or near real-time. Alternatively, or additionally, the GUI 500 may include a threshold optimization element 526 which is selectable to automatically determine threshold value(s) that maximize relevant data output of a particular waveform data set.

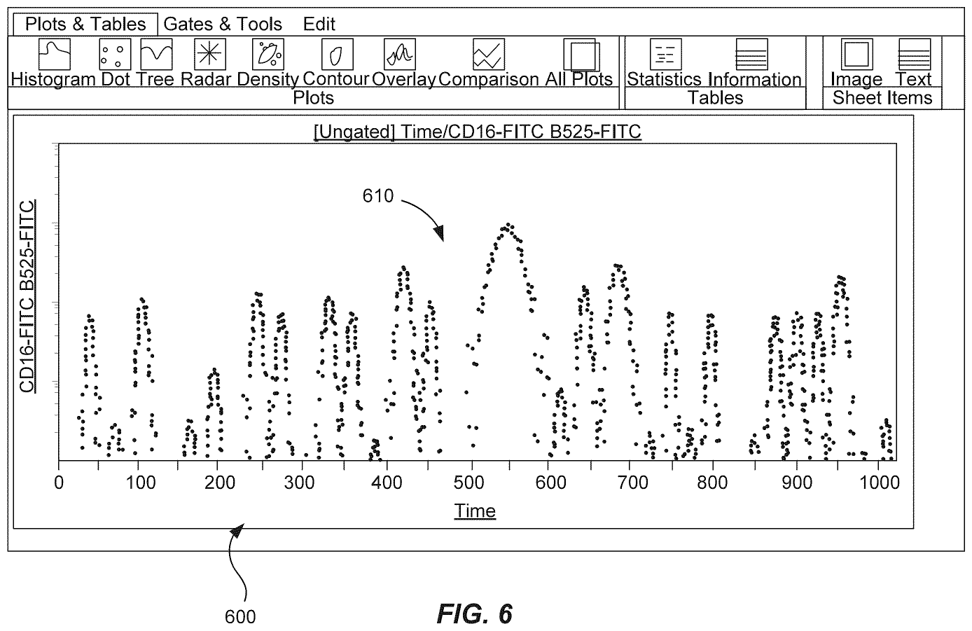

FIG. 6 shows an example of a waveform display window 600 graphically displaying waveform data 610 that is unthresholded. That is, in contrast to display windows of previous cytometer analysis devices that show the computed height, width, and area values (i.e., event data) obtained from an FPGA, the waveform analysis device 150 described herein displays the waveform data 610 in its entirety and in digital form. By displaying the waveform data 610 in this form, a technical benefit is provided in that the user can visually see the features of pulses and noise to determine the appropriate threshold level for measuring height, width, and area during post-processing as opposed to calculating the parameters during acquisition.

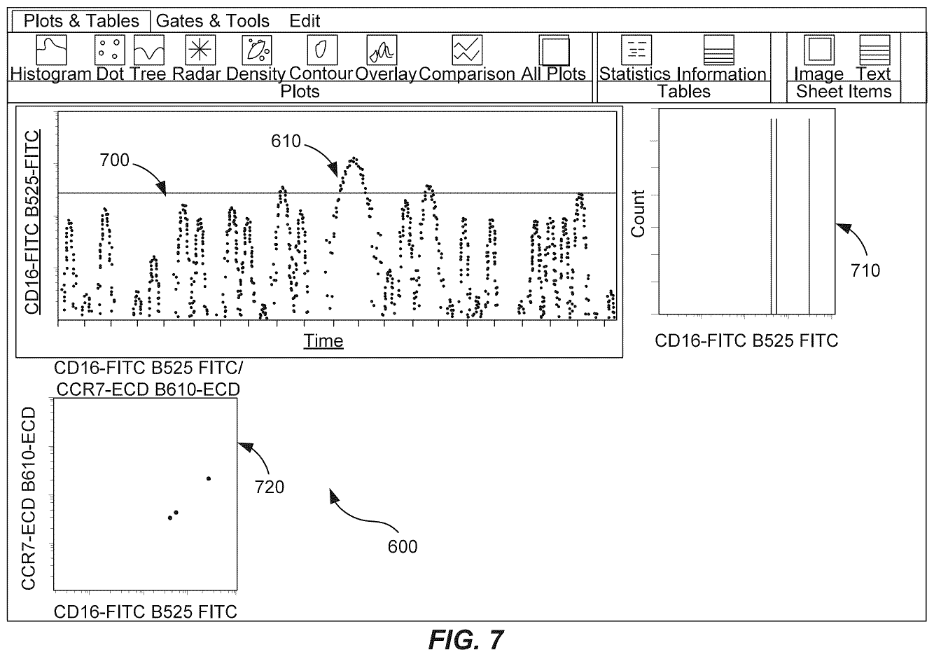

FIG. 7 shows an example of a high threshold value 700 applied to the waveform data 610 in the waveform display window 600. As the high threshold value 700 begins to intersect with the highest pulse peaks of the waveform data 610, the GPU 152 may extract height, width, and area and update the graphs and plots in the waveform display window 600 in real-time. In this example, the waveform display window 600 includes a histogram 710 and scatter plot 720 which each display a few extracted measurements due to the minimal amount of intersection between the high threshold value 700 and the waveform data 610.

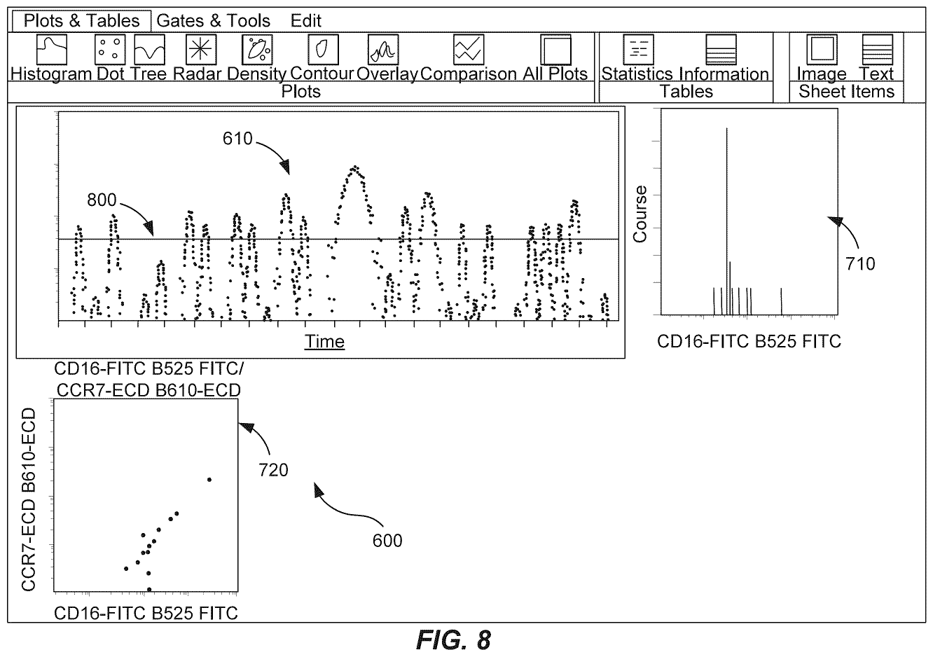

FIG. 8 shows an example of an intermediate threshold value 800 applied to the waveform data 610 in the waveform display window 600. Compared with the high threshold value 700, the intermediate threshold value 800 intersects with an increased number of pulses in the waveform data 610. Accordingly, the histogram 710 is updated in real-time to display additional bars indicating the count of a particular type of cell detected. Similarly, the scatter plot 720 is updated in real-time to display additional dots reflecting the additional particles measured and their detected relative fluorescence intensity of two parameters plotted on its two axes.

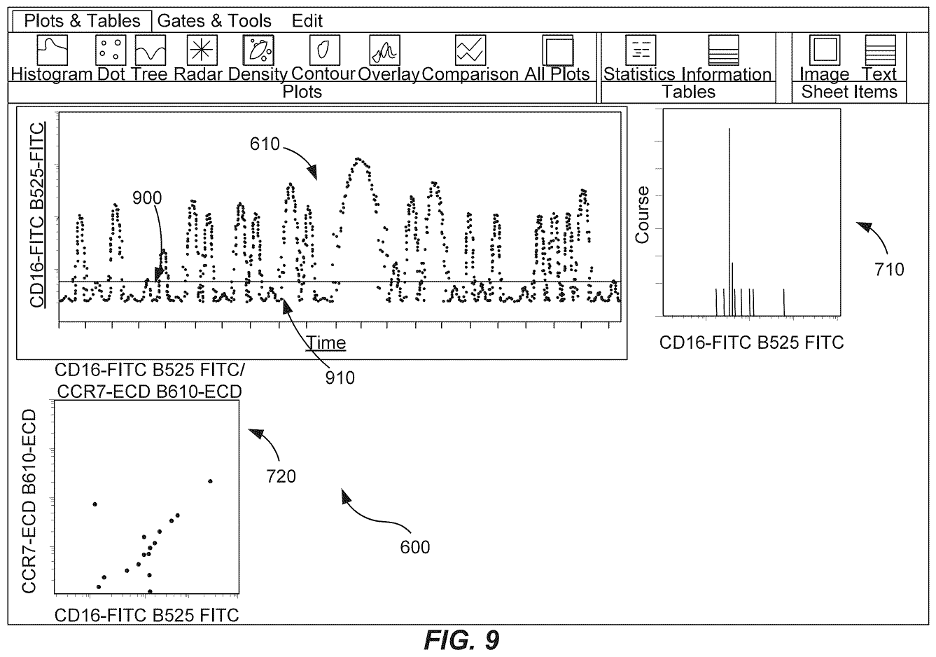

FIG. 9 shows an example of a low threshold value 900 applied to the waveform data 610 in the waveform display window 600. The GPU 152 continues to measure and extract event data from the waveform data 610 in real-time as the threshold value is lowered and the histogram 710 and scatter plot 720 are updated accordingly. In this example, the low threshold value 900 may be set to a value immediately above noise 910 that is visible in the waveform data 610. The noise 910 may therefore be filtered out based on visual features of the waveform data 610. In some embodiments, the GUI 500 allows the user to apply a logical complement of a threshold. For example, suppose a researcher is interested in studying the noise 910 in the waveform data 610 of a cytometry experiment. In such instance, the researcher can set the low threshold value 900 to extract data having voltage values below the low threshold value 900 instead of above it.

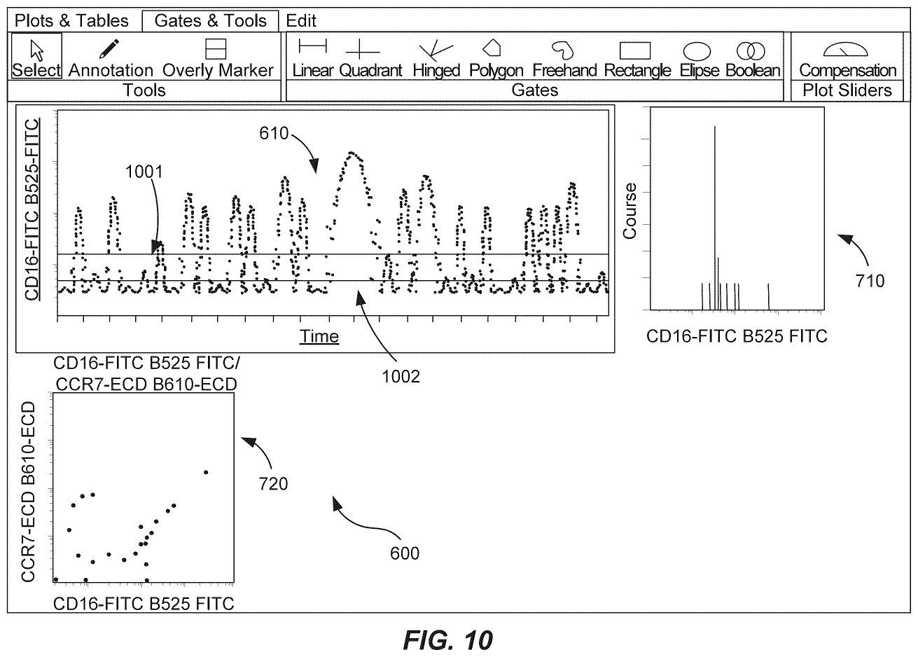

FIG. 10 shows an example of multiple thresholds applied to the waveform data 610 in the waveform display window 600. For example, when identifying extracellular vesicles (EVs), which are cells typically larger than noise and smaller than other particles of a sample, the GUI 500 enables application and adjustment of first and second thresholds 1001, 1002 to extract event data in a region between the thresholds. That is, the region may be defined as digital values in the waveform data 610 that are below the first threshold 1001 and above the second threshold 1002. In some examples, the waveform analysis device 150 is configured to determine a first threshold voltage comprising a threshold function that is smaller than peaks of a largest group of pulses of the waveform data 610, to determine a second threshold voltage comprising another threshold function that is larger than noise of the waveform data 610, and to analyze the waveform data 610 with respect to the first threshold voltage and the second threshold voltage to extract event data. This approach can be used to detect and analyze nanoparticles in a sample such as EVs.

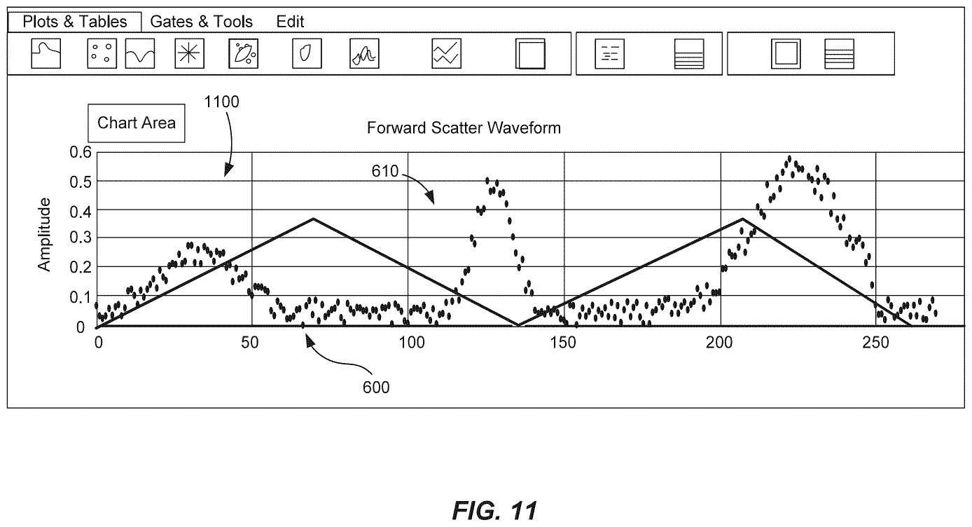

FIG. 11 shows an example of a non-linear threshold 1100 applied to the waveform data 610 in the waveform display window 600. Since thresholding is applied by the GPU 152 during post-processing, one or more non-linear thresholds 1100 may be applied to the waveform data 610. In some embodiments the threshold values are non-constant. The non-linear thresholds 1100 are examples of one or more threshold values that are non-constant. In this example, the non-linear threshold 1100 is a sawtooth signal, though other non-linear or non-constant thresholds may comprise any number of functions such as sinusoidal functions, step functions, and the like. In some embodiments, the GPU 152 determines the threshold function automatically based on curvature of the pulses of the waveform data 610. For example, the non-linear threshold 1100 may comprise a first or second derivative of one or more pulses in the waveform data 610. In some embodiments, a non-linear threshold 1100 may be determined for each pulse to generate a moving function for the waveform data 610. The GPU 152 may then extract values in the area underneath the non-linear threshold(s) 1100. In some embodiments, thresholds may be applied to waveform data in a domain other than time, such as frequency. For instance, the GPU 152 may apply a threshold as a fast Fourier transform (FFT) applied to the waveform data 610 to generate vector valued data for each of the particles.

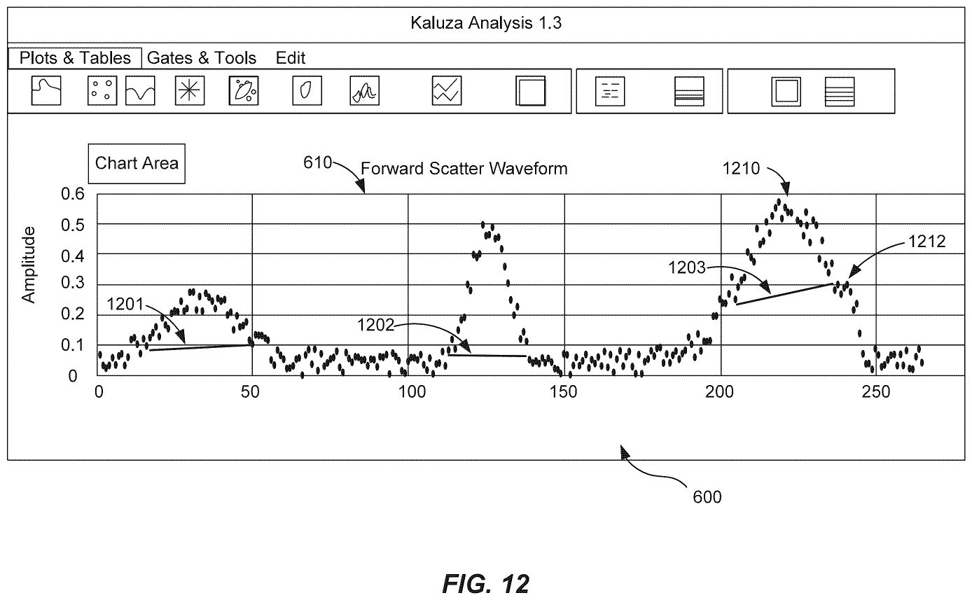

FIG. 12 shows an example of event-specific thresholds 1201-1203 applied to the waveform data 610 in the waveform display window 600. In some embodiments, the cytometry analysis application 450 (see FIG. 4) determines thresholds on a per-cell basis. For example, each pulse in the waveform data 610 may be assigned a threshold that maximizes the data output of each particle while minimizing noise. The event-specific thresholds 1201-1203 may be horizontal, slanted, and/or non-linear thresholds that each apply to a specific event within the waveform data 610 for a discrete period of time. For example, a user or the cytometry analysis application 450 can determine to filter out data points 1212 of a particular pulse 1210 of the waveform data 610. An event-specific threshold 1203 of the pulse 1210 may be adjusted with a right end at an upward slant such that the data points 1212 are not used for analysis or plotting. The adjustment may be performed by the user selecting and dragging the right end of the event-specific threshold 1203 or be automatically adjusted by an algorithm executed by the cytometry analysis application 450.

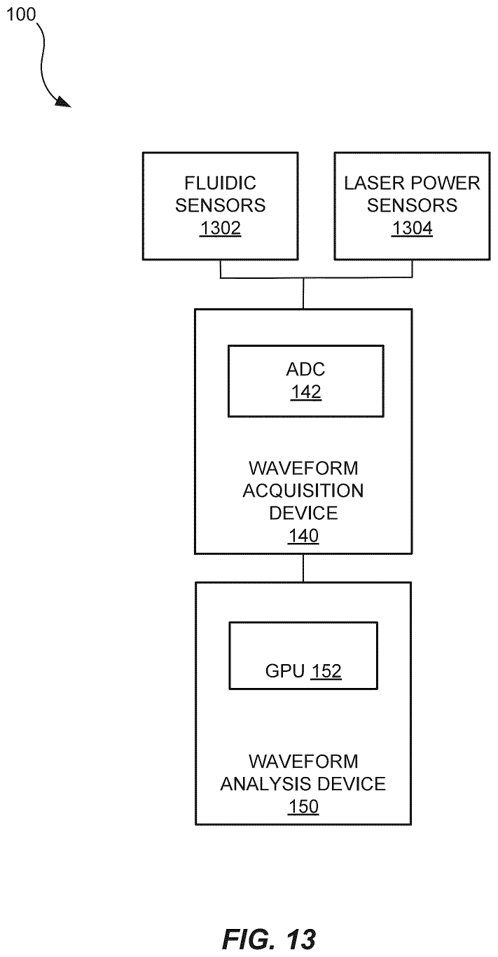

FIG. 13 is a block diagram of another example of the flow cytometer system 100. In addition to the digitized waveform data output by the detectors 124, the waveform analysis device 150 of some embodiments may collect, analyze, and display digitized data of other sensors in the flow cytometer system 100 including one or more fluidic sensors 1302 and/or one or more laser power sensors 1304. A fluidic sensor 1302 may output measurements of a sheath pressure of the nozzle 112 as particles eject. A laser power sensor 1304 may output measurements indicating variation in a power or intensity in the output of the laser 102 as particles are interrogated. In some embodiments, an analog-to-digital converter 142 of the waveform acquisition device 140 digitizes the measurements before reception at the waveform analysis device 150.

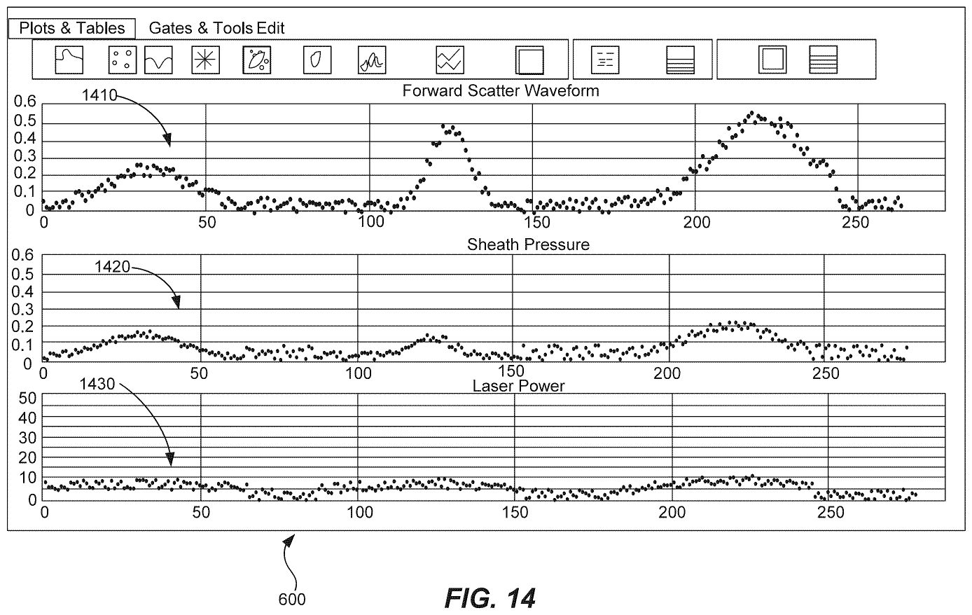

FIG. 14 shows an example of the waveform display window 600 graphically displaying waveform plots for data collected by the flow cytometer system 100 of FIG. 13. In this example, the waveform plots include forward scatter waveform data 1410 output by a detector 124 of the optical system 120, sheath pressure waveform data 1420 output by a fluidic sensor 1302, and laser power waveform data 1430 output by a laser power sensor 1304. The waveform plots may be displayed in alignment with respect to time such that a user may visually observe multiple measurements corresponding with a particular particle in an experiment. The sheath pressure waveform data 1420 and the laser power waveform data 1430 respectively provided by the fluidic sensor 1302 and laser power sensor 1304 may be used, for example, to set and adjust one or more thresholds applied to the forward scatter waveform data 1410. This measurement data may also be used to standardize instruments of the flow cytometer system 100.

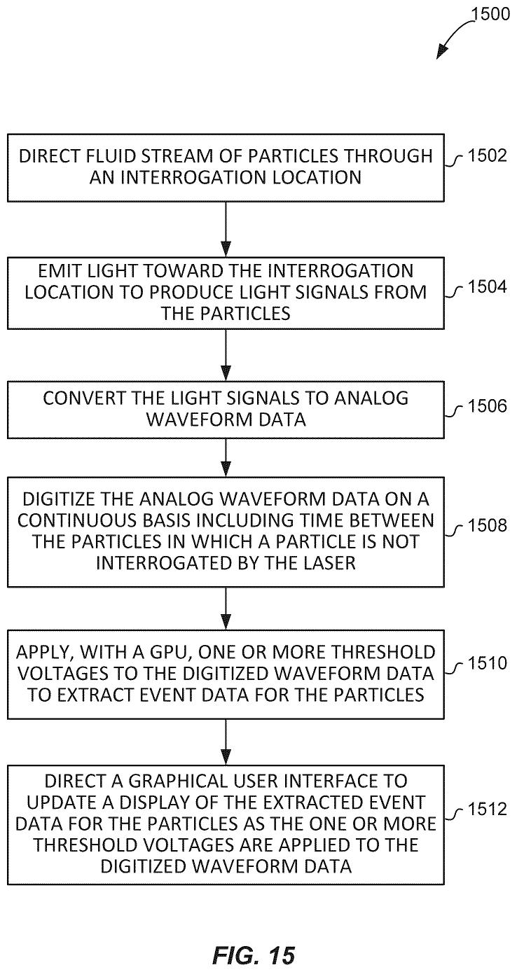

FIG. 15 is a flowchart illustrating an example of a method 1500 of analyzing particles in the flow cytometer system 100. In step 1502, a fluid stream of particles is directed through the interrogation location 116. In step 1504, laser light is directed toward the interrogation location 116 to produce emitted light signals from the particles. In step 1506, the emitted light signals are converted to raw/analog waveform data. In step 1508, the analog data is digitized on a continuous basis. In step 1510, the GPU 152 applies one or more threshold voltages to the digitized waveform data to extract event data for the particles. In some examples, the threshold may be applied after storing the waveform data in the persistent storage 430 of the waveform analysis device 150. In step 1512, the GUI 420 is directed to update a display of the extracted event data for the particles as the one or more threshold voltages are applied to the digitized waveform data. One or more linear, logarithmic, and/or logicle display techniques may be applied to format the display of event data. In some examples, statistics are computed for the event data. For example, the GPU 152 may process the event data to compute one or both of skew and kurtosis for each of the particles. The steps 1510 and 1512 may be repeated on the digitized waveform data in response to changes in thresholds or other settings without requiring the sample to be reprocessed and reinterrogated by steps 1502, 1504, 1506, and 1508.

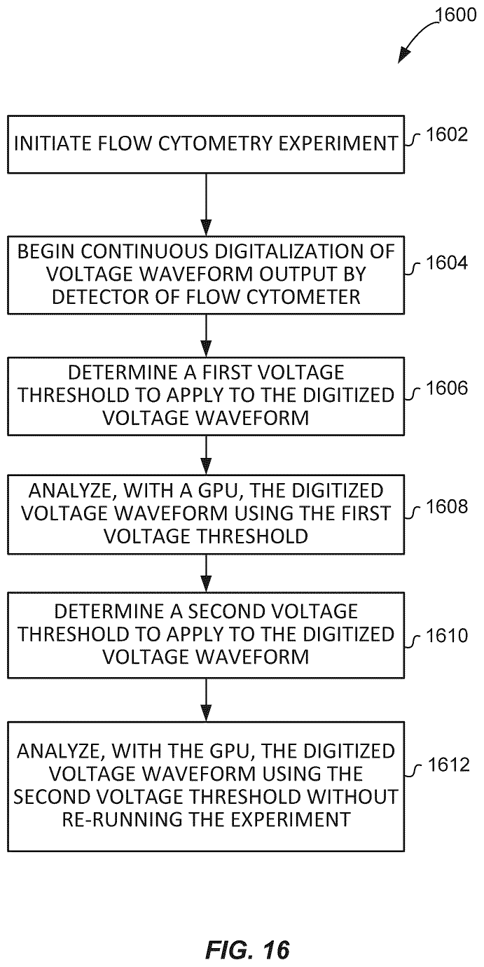

FIG. 16 is a flowchart illustrating another example of a method 1600 of analyzing particles in the flow cytometer system 100. In step 1602, a flow cytometry experiment is initiated. In step 1604, continuous digitalization of a voltage waveform output by a detector 124 of the flow cytometer 101 begins. In step 1606, a first voltage threshold is determined to apply to the digitized voltage waveform. In step 1608, the GPU 152 analyzes the digitized voltage waveform using the first voltage threshold. In step 1610, a second voltage threshold is determined to apply to the digitized voltage waveform. In step 1612, the GPU 152 analyzes the digitized voltage waveform using the second voltage threshold without re-running the experiment.

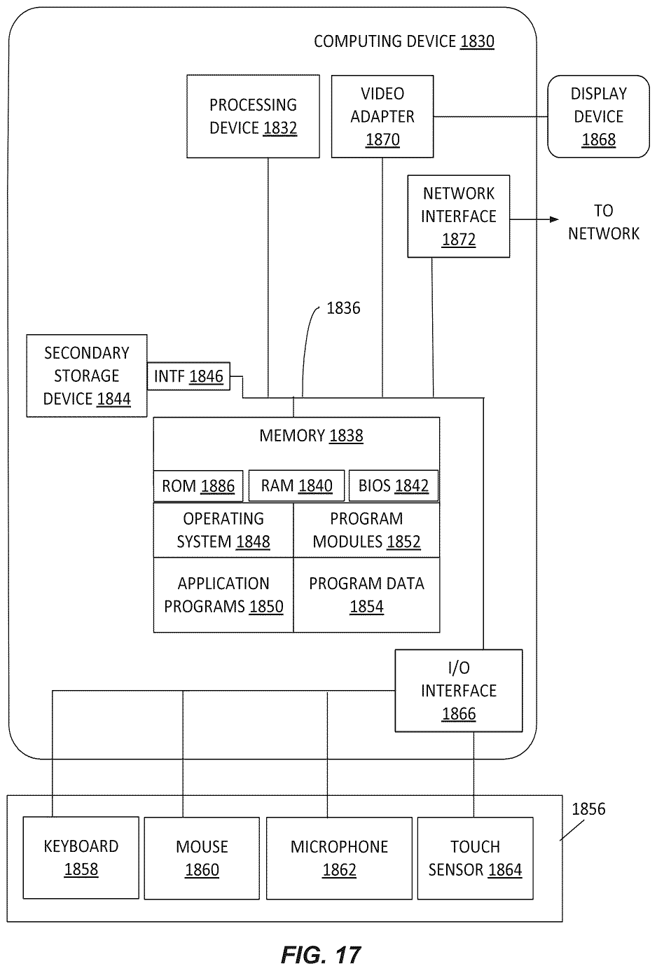

FIG. 17 illustrates an exemplary architecture of a computing device 1830 that can be used to implement aspects of the flow cytometer system 100, including the waveform analysis device 150. The computing device 1830 can be used to execute the operating system, application programs, and software modules (including the software engines) described herein. Examples of computing devices suitable for the computing device 1830 include a server computer, a desktop computer, a laptop computer, a tablet computer, a mobile computing device (such as a smartphone), or other devices configured to process digital instructions.

The computing device 1830 includes at least one processing device 1832, such as a central processing unit (CPU). A variety of processing devices are available. The computing device 1830 also includes a system memory 1838, and a system bus 1836 that couples various system components including the system memory 1838 to the at least one processing device 1832. The system bus 1836 is one of any number of types of bus structures including a memory bus, or memory controller; a peripheral bus; and a local bus using any of a variety of bus architectures.

The system memory 1838 can include a read only memory (ROM) 1886 and a random access memory (RAM) 1840. A basic input/output system (BIOS) 1842 containing the basic routines that act to transfer information within computing device 1830, such as during start up, can be stored in the system memory 1838. The waveform analysis device 150 can have a large memory capacity, such as equal to or greater than one Terabyte of RAM. The RAM 1840 can be used by the GPU 152 for loading and subsequently analyzing the waveform data (e.g., the raw waveform data stored in a raw waveform data file, which can include digitalized waveform data).

The computing device 1830 can also include a secondary storage device 1844 such as a hard disk drive for storing digital data. The secondary storage device 1844 is connected to the system bus 1836 by a secondary storage interface 1846. The secondary storage devices 1844 and associated computer readable media provide nonvolatile storage of computer readable instructions (including application programs and program modules), data structures, and other data for the computing device 1830. Although the example described herein employs a hard disk drive as a secondary storage device, other types of computer readable storage media are used in other embodiments. Examples of these other types of computer readable storage media include the RAM 1840 and/or the ROM 1886. Some examples include non-transitory media. Additionally, such computer readable storage media can include local storage or cloud-based storage.

The computing device 1830 typically includes at least some form of computer readable media. Computer readable media includes any available media that can be accessed by the computing device 1830. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, or any other medium that can be used to store the desired information and that can be accessed by the computing device 1830.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

A number of program modules can be stored in secondary storage device 1844 or the system memory 1838, including an operating system 1848, application programs 1850, program modules 1852 (such as the software engines), and program data 1854. The computing device 1830 can utilize any suitable operating system, such as Microsoft Windows™, Google Chrome™, Apple OS, and any other operating system suitable for a computing device.

A user provides inputs to the computing device 1830 through one or more input devices 1856. Examples of input devices 1856 include a keyboard 1858, a mouse 1860, a microphone 1862, and a touch sensor 1864 (such as a touchpad or touch sensitive display). Additional types of the input devices 1856 are contemplated. The input devices 1856 are often connected to the at least one processing device 1832 through an input/output interface 1866 that is coupled to the system bus 1836. These input devices 1856 can be connected by any number of input/output interfaces, such as a parallel port, serial port, game port, or a universal serial bus. Wireless communication between input devices and the input/output interface 1866 is possible as well, and includes infrared, BLUETOOTH® wireless technology, 802.11a/b/g/n, cellular, or other radio frequency communication systems in some possible embodiments.

A display device 1868, such as a monitor, liquid crystal display device, projector, or touch sensitive display device, can also be connected to the system bus 1836 via an interface, such as a video adapter 1870. In addition to the display device 1868, the computing device 1830 can include various other peripheral devices (not shown), such as speakers or a printer.

When used in a local area networking environment or a wide area networking environment (such as the Internet), the computing device 1830 is typically connected to a network through a network interface 1872, such as an Ethernet interface. Other possible embodiments use other communication devices. For example, some embodiments of the computing device 1830 include a modem for communicating across the network.

The computing device 1830 is an example of programmable electronics, which may include one or more such computing devices, and when multiple computing devices are included, such computing devices can be coupled together with a suitable data communication network so as to collectively perform the various functions, methods, or operations disclosed herein.

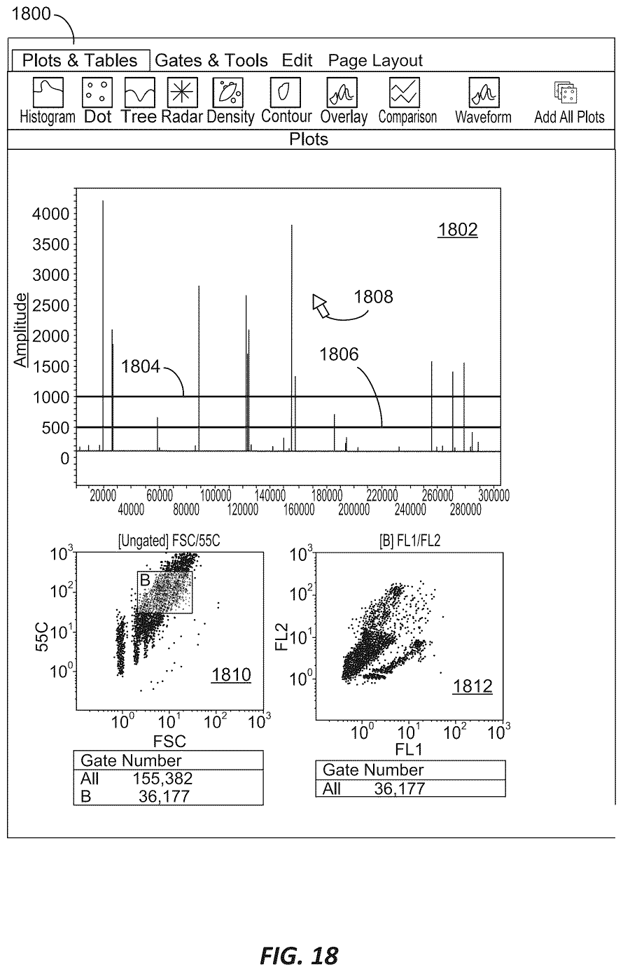

FIG. 18 illustrates another example of a graphical user interface (GUI) 1800 that can be generated by the waveform analysis device 150. The GUI 1800 includes a waveform plot 1802, an upper threshold slider 1804, and a lower threshold slider 1806. In the example shown in FIG. 18, the upper threshold slider 1804 is positioned at 1000 amplitude units, and the lower threshold slider 1806 is positioned at 500 amplitude units. Additionally, the GUI 180 includes first and second scatter plots 1810, 1812 that display events extracted from the waveform plot 1802 based on the positions of the upper and lower threshold sliders 1804, 1806.

The GUI 1800 further displays a mouse pointer 1808 that is controlled by operating the mouse 1860 to move about the GUI 1800 and to select one or more selectable icons on the GUI 1800. Additional examples of user interfaces on the waveform analysis device 150 are contemplated such that the mouse pointer 1808 and the mouse 1860 are provided by way of illustrative example. In this example, a user of the waveform analysis device 150 can move the mouse 1860 to hover over the upper threshold slider 1804 and can click the mouse 1860 to select the upper threshold slider 1804. While the upper threshold slider 1804 is selected, the user can move the upper threshold slider 1804 up or down to adjust the value of the upper threshold slider 1804. Similarly, a user of the waveform analysis device 150 can move the mouse 1860 to hover over the lower threshold slider 1806 and can click the mouse 1860 to select the lower threshold slider 1806. While the lower threshold slider 1806 is selected, the user can move the lower threshold slider 1806 up or down to adjust the value of the lower threshold slider 1806.

As the upper or lower threshold sliders 1804, 1806 are adjusted, the events extracted from the waveform plot 1802 displayed in the first and second scatter plots 1810, 1812 are adjusted. As discussed above, the waveform acquisition device 140 continuously digitizes the outputs from the detectors 124 regardless of whether a cell is currently interrogated by the laser 102. This generates a stream of data that is approximately 1000 times larger (e.g., at a 1 GHz sampling rate) than state of the art waveform acquisition devices that use a single threshold value to determine when the outputs from the detectors are converted from analog to digital.

Table 1 summarizes performance of updating the GUI 1800 based on adjustments of the upper or lower threshold slider 1804, 1806. In Table 1, the first column includes the number of data points (in millions) in each of four waveforms. The four waveforms include a forward scatter waveform, a side scatter waveform, and fluorescence waveforms that result from energy emitted by fluorescent dyes when stimulated by the laser 102. The second column in Table 1 includes run times for updating the GUI 1800 when the data points for the four waveforms are processed by a single central processing unit (CPU) core, the third column in Table 1 includes run times for updating the GUI 1800 when the data points for the four waveforms are processed by four CPU cores, and the fourth column in Table 1 includes run times for updating the GUI 1800 when the data points for the four waveforms are processed by the GPU 152.

TABLE 1

| Number of | Speed with 1 | Speed with 4 | |
|---|---|---|---|
| Waveform Points | CPU Core | CPU Cores | GPU Speed |
| (millions) | (seconds) | (seconds) | (seconds) |
| 500 | 2.9 | 1.6 | 0.029 |
| 1,000 | 5.6 | 3.2 | 0.060 |
| 2,000 | 11.5 | 6.6 | 0.120 |
| 4,000 | 23.2 | 12.0 | |
| 8,000 | 50.2 | 28.5 | |

As shown in Table 1, when either the upper or lower threshold slider 1804, 1806 is moved, the time for updating the GUI 1800 using a single CPU core when there are 500 million data points for each of the four waveforms is 2.9 seconds. The time for updating the GUI 1800 using four CPU cores when there are 500 million data points for each of the four waveforms is reduced to 1.6 seconds. The time for updating the GUI 1800 using the GPU 152 when there are 500 million data points for each of the four waveforms is 0.029 seconds.

In the second row of Table 1, the number of waveform points per waveform doubles (1 billion waveform points), and as a result, the run times of the 1 CPU core, 4 CPU core, and GPU embodiments, in general, approximately double as well. Similarly, in the third row of Table 1, the number of waveform points per waveform doubles (2 billion waveform points), and the run times of the 1 CPU core, 4 CPU core, and GPU embodiments, approximately double again.

The fourth row of Table 1 shows that the 4 CPU core embodiment runs at 12 seconds for updating the GUI 1800 when there are 4 billion waveform points, and the fifth row of Table 1 shows that the 4 CPU core embodiment runs at 28.5 seconds for updating the GUI 1800 when there are 8 billion waveform points. Such response times do not provide an interactive experience such as one where the GUI 1800 is updated in real-time or in near real-time.

Further, the run times of the GPU embodiment are non-existent in the fourth and fifth rows of Table 1. This is because the waveform points of the fourth and fifth rows no longer fit within a random-access memory (RAM) of the GPU 152. Typically, GPUs include a maximum of 80 GB of RAM memory onboard. When more than the maximum capacity of data available on the RAM of the GPU 152 is required to update the GUI 1800, the waveform points are truncated and the GPU 152 cannot properly update the GUI 1800. Given the foregoing, an interactive experience for updating the GUI 180 is limited by the capacity of the RAM memory of the GPU 152, while on the other hand, performance is unacceptable when utilizing the 1 CPU core and 4 CPU core embodiments for large data set sizes of waveform points.

As will now be described in more detail, a hybrid technique can be implemented on the flow cytometer system 100 that utilizes a host random access memory (RAM) for storage of the waveform data and the GPU 152 for processing of the waveform data. The hybrid technique can significantly reduce the response time for calculating one or more parameters and displaying events in the first and second scatter plots 1810, 1812 in response to the adjustments of the upper and lower threshold sliders 1804, 1806 in the GUI 1800.

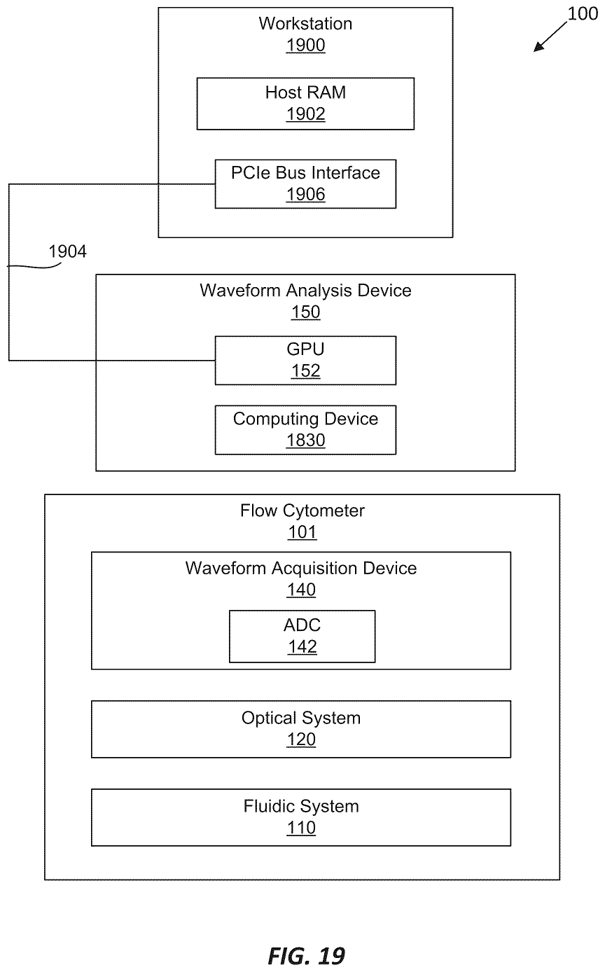

FIG. 19 schematically illustrates an example of the flow cytometer system 100 that includes a workstation 1900 having a host random access memory (RAM) 1902. Examples of the workstation 1900 include a desktop computer, a personal computer (PC), and the like. In further examples, the host RAM 1902 can be housed on a server such as a remote server or a cloud server. As an example, the host RAM 1902 has a memory capacity of 1 terabyte, or more.

As shown in FIG. 19, the host RAM 1902 is communicatively connected to the graphics processing unit 152 via a connection 1904. In some examples, the connection 1904 is a physical connection such as a cable that physically connects the host RAM 1902 to the waveform analysis device 150. In such examples, the physical connection can be accomplished through wired computer networking technologies such as Ethernet. Alternatively, the connection 1904 can include a wireless connection such as one accomplished through of wireless network protocols including satellite communication networks, cellular networks, Wi-Fi, and the like.

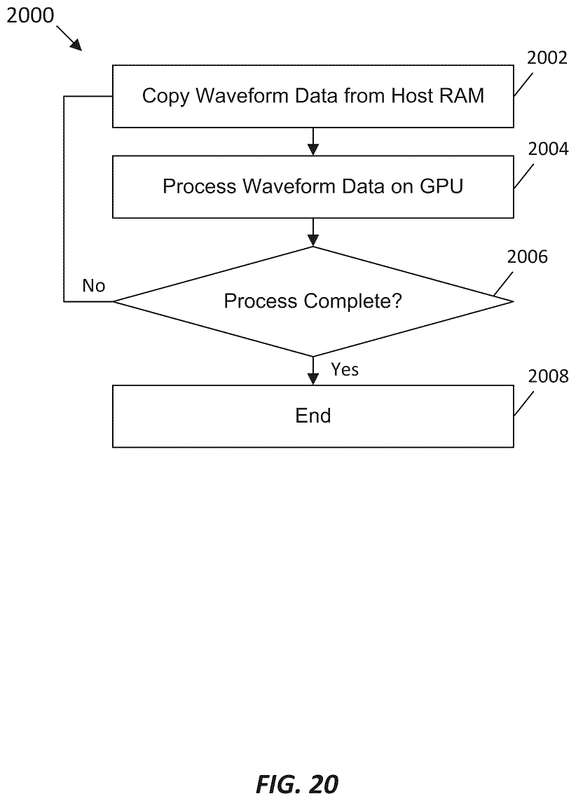

In some examples, communications between the host RAM 1902 and the waveform analysis device 150 can be accomplished by using a peripheral component interconnect express (PCIe) bus interface 1906 housed on the workstation 1900. The PCIe bus interface 1906 is a high-speed serial computer expansion bus standard that provides a motherboard interface for graphics cards, sound cards, hard disk drive host adapters, solid-state drives (SSDs), Wi-Fi, and Ethernet hardware connections on the workstation 1900. As an illustrative example, the PCIe bus interface 1906 can transfer data to and from the workstation 1900 at 64 GB per second. FIG. 20 schematically illustrates an example of a method 2000 of performing the hybrid technique to update the GUI 1800 based on adjustments of the upper or lower threshold slider 1804, 1806. In the method 2000, the waveform data is stored in the host RAM 1902.

The method 2000 includes a step 2002 of copying waveform data from the host RAM 1902 to the GPU 152. As an illustrative example, step 2002 can include copying 32 GB of waveform data from the host RAM 1902 to the GPU 152. As a further example, transferring the 32 GB of waveform data from the host RAM 1902 to the GPU 152 takes about 0.5 seconds.

Next, the method 2000 includes a step 2004 of processing the waveform data on the GPU 152. As an illustrative example, processing 4×2000 m waveform points on the GPU 152 takes about 0.120 seconds (see Table 1, third row).

Next, the method 2000 includes a step 2006 of determining whether all of the waveform data points have been processed. When it is determined that not all waveform data points have been processed (i.e., “No” in step 2006), the method 2000 proceeds to repeat steps 2002-2006. Otherwise, when all the waveform data points have been processed (i.e., “Yes” in step 2006), the method 2000 terminates at step 2008.

Table 2 summarizes performance of updating the GUI 1800 based on the method 2000. As shown in Table 2, when there are 4 billion data points for each of the four waveforms, the total run time is 0.740 seconds, which is calculated by the time to transfer the waveform data from the host RAM 1902 to the GPU 152 (0.500 seconds) plus the time required by the GPU 152 to process the waveform data (2×0.120 seconds). This run time is significantly less the run time of 12 seconds required by the four CPU core embodiment (see Table 1).

TABLE 2

| Number of Waveform | Hybrid Technique |
|---|---|
| Points (millions) | (seconds) |
| 2,000 | 0.120 |
| 4,000 | 0.740 |
| 8,000 | 1.480 |

The 8 billion data points for each of the four waveforms represents a data size of 64 GB. Thus, processing this waveform data requires two waveform data transfers (e.g., each being 32 GB) from the host RAM 1902 to the GPU 152 at 0.5 seconds each for a total time of 1 second. The time required by the GPU 152 to process the waveform data is 4×0.120 seconds, thus providing a total run time of 1.480 seconds. This run time is significantly less the run time of 28.5 seconds required by the four CPU core embodiment (see Table 1).

Additionally, the run times for updating the GUI 1800 based on the method 2000 can be further reduced by improving the performance of the PCIe bus interface 1906. For example, the run times shown in Table 2 are based on a PCIe-4 bus interface that transfers data to and from the workstation 1900 at 64 GB per second (i.e., 0.5 seconds per 32 GB transfer). Alternatively, when a PCIe-5 bus interface is used, the data transfer rate is 128 GB per second. Thus, copying 32 GB of waveform data from the host RAM 1902 to the GPU 152 would take 0.25 seconds instead of 0.5 seconds. Table 3 summarizes the performance of updating the GUI 1800 based on the method 2000 when PCIe-5 bus interface is used.

TABLE 3

| Number of Waveform | Hybrid Technique |
|---|---|
| Points (millions) | (seconds) |
| 2,000 | 0.120 |
| 4,000 | 0.490 |
| 8,000 | 0.980 |

Another technique to even further reduce the run times for updating the GUI 1800 based on adjustments of the upper or lower threshold slider 1804, 1806 can include compressing the waveform data. For example, a lossless compression algorithm can provide a 2× compression such that the transfer of N bytes of waveform data in T time units, would result in 2N bytes being present on the GPU after the transfer and subsequent decompression.

In addition to the hybrid technique described above, a data decimation technique can also be implemented to further enhance the interactive experience of a user of the flow cytometer system 100. The data decimation technique can trick the user into thinking the GUI 1800 is being updated in real-time or near real-time by decimating the waveform data when the user clicks the mouse pointer 1808 on one of the upper and lower threshold sliders 1804, 1806.

As an illustrative example, when the waveform data includes 8 billion data points for each waveform of the four waveforms (i.e., row 5 of Table 1), and when the user clicks the mouse pointer 1808 on one of the upper and lower threshold sliders 1804, 1806, the data decimation technique includes processing a subset of the waveform data points such as 1 of every 16 waveform data points. For example, as the user moves the upper threshold slider 1804 or the lower threshold slider 1806, the GPU 152 processes a data set size that corresponds to 500 million data points for each waveform of the four waveforms (i.e., row 1 of Table 1). Thus, the event extraction by the GPU 152 for the four waveforms will happen in 0.029 seconds. As the events are extracted, the histograms, scatter plots, density plots, and the like displayed in the GUI 1800 will update in real-time or near real-time giving the user an interactive experience.

When the user is satisfied with a threshold value associated with the upper threshold slider 1804 or lower threshold slider 1806, the user unclicks the mouse pointer 1808 on the upper threshold slider 1804 or lower threshold slider 1806. At that point, the GPU 152 processes all of the waveform data points (8 billion data points for each of the four waveforms) with a run time of 1.480 seconds (see row 3 of Table 2) or 0.980 seconds (see row 3 of Table 3) depending on the PCIe bus interface used for transferring the data from the host RAM 1902. Given the foregoing, the data decimation technique includes processing a subset of the waveform data while the upper threshold slider 1804 or lower threshold slider 1806 is being moved by the user, and thereafter processing all of the waveform data when the user lets up on the mouse 1860.

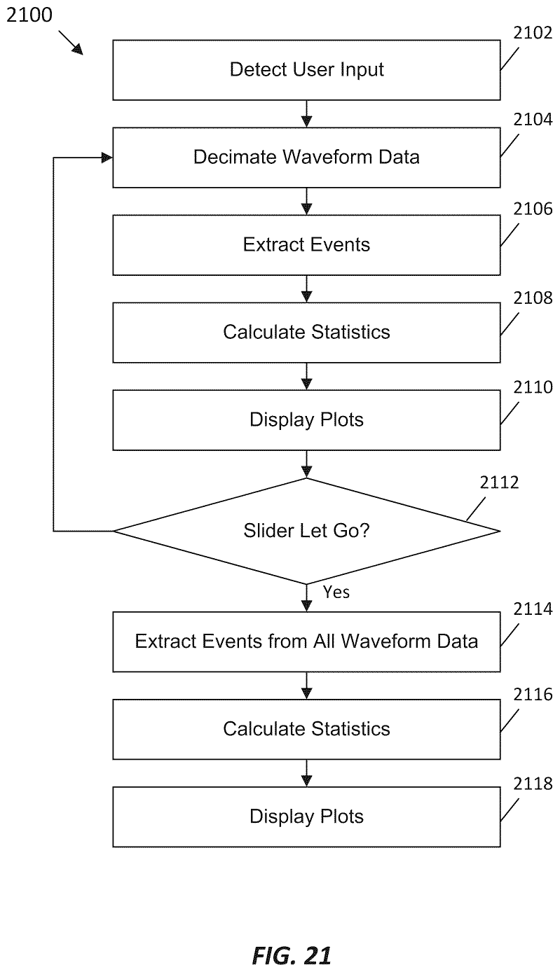

FIG. 21 schematically illustrates an example of a method 2100 of decimating waveform data that can be performed by the waveform analysis device 150. The method 2100 includes a step 2102 of detecting a user input on the upper threshold slider 1804 or lower threshold slider 1806. The user input can include the mouse pointer 1808 selecting the upper threshold slider 1804 or lower threshold slider 1806, and then moving the upper threshold slider 1804 or lower threshold slider 1806 up or down to adjust its value.

The method 2100 includes a step 2104 of decimating the waveform data points to a subset of waveform data points. Step 2104 can include decimating the waveform data points by a ratio such as 1:32, 1:16, 1:8, and the like. For example, when there are 8 billion waveform data points, step 2104 can include decimating the waveform data points by 1:16 to provide a subset of 500 million waveform data points. Additional examples are contemplated.

The method 2100 includes a step 2106 of extracting events (e.g., waveform data points) based on a current value of the upper threshold slider 1804 or lower threshold slider 1806, and the decimation of the waveform data points performed in step 2104. For example, when one of the upper and lower threshold sliders 1804, 1806 is being moved while the other is being stationary, step 2106 includes extracting events that have values between the moving or stationary upper threshold slider 1804 and the moving or stationary lower threshold slider 1806.

The method 2100 includes a step 2108 of calculating one or more statistics based on the events extracted in step 2106. Step 2108 includes calculating statistics based on the subset of waveform data points instead of the total number of waveform data points that are extracted based on the current values of the upper and lower threshold sliders 1804, 1806. For example, step 2108 can include calculating a minimum value, a maximum value, an average value, and a mode value from the subset of waveform data points.

The method 2100 includes a step 2110 of updating one or more displays of data based on the events extracted in step 2106. For example, step 2110 can include updating histograms, scatter plots, density plots, and the like based on the subset of waveform data points instead of the total number of waveform data points that are extracted based on the current values of the upper and lower threshold sliders 1804, 1806.

The method 2100 includes a step 2112 of determining whether the mouse pointer 1808 is unclicked such that the upper threshold slider 1804 or the lower threshold slider 1806 is no longer selected. When it is determined that the mouse pointer 1808 remains clicked such that the upper threshold slider 1804 or the lower threshold slider 1806 remains selected (i.e., “No” in step 2112), the method 2100 can repeat steps 2102-2112. Otherwise, when it is determined that the mouse pointer 1808 is unclicked such that the upper threshold slider 1804 or the lower threshold slider 1806 is no longer selected (i.e., “Yes” in step 2112), the method 2100 proceeds to a step 2114 of extracting all of the waveform data points based on current values of the upper threshold slider 1804 and the lower threshold slider 1806.

The method 2100 includes a step 2116 of calculating one or more statistics based on the events extracted in step 2114. For example, step 2116 can include calculating statistics based the total number of waveform data points instead of the subset of waveform data points that are extracted based on the current values of the upper and lower threshold sliders 1804, 1806.

The method 2100 includes a step 2118 of updating one or more displays of data based on the events extracted in step 2114. For example, step 2118 can include updating histograms, scatter plots, density plots, and the like based on the total number of waveform data points that are extracted based on the current values of the upper and lower threshold sliders 1804, 1806.

The method 2100 provides a data decimation technique that improves the interactive experience provided by the waveform analysis device 150 by more quickly updating the display of statistics and plots on the GUI 1800 by processing a subset of the waveform data when the upper threshold slider 1804 or lower threshold slider 1806 is being selected and moved by the user. When the user lets up on the mouse 1860 such as when the user is satisfied with a value of the upper threshold slider 1804 or lower threshold slider 1806, the method 2100 updates the display of statistics and plots on the GUI 1800 by processing all of the waveform data.

Although specific embodiments are described herein, the scope of the disclosure is not limited to those specific embodiments. The scope of the disclosure is defined by the following claims and any equivalents thereof.

1. A flow cytometry system configured to direct a fluid stream of particles through an interrogation location, the flow cytometry system comprising:
a laser configured to emit light toward the interrogation location to produce light signals from the particles;
one or more detectors configured to convert the light signals to waveform data;
a waveform acquisition device configured to digitize the waveform data; and
a graphics processing unit configured to apply one or more adjustable threshold voltages to the digitized waveform data to extract event data from the particles.

2. The flow cytometry system of claim 1, wherein the graphics processing unit is configured to direct a graphical user interface to update a display of the event data as the one or more adjustable threshold voltages are applied.

3. The flow cytometry system of claim 1, further comprising:
a waveform analysis device including the graphics processing unit, the waveform analysis device configured to apply the one or more adjustable threshold voltages.

4. The flow cytometry system of claim 3, wherein the waveform analysis device is configured to apply the one or more adjustable threshold voltages based on movement along a scale to increase or decrease the one or more adjustable threshold voltages.

5. The flow cytometry system of claim 4, wherein in response to the movement along the scale to increase or decrease the one or more adjustable threshold voltages, the graphics processing unit is configured to generate updated event data for the particles without re-running the particles through the interrogation location.

6. The flow cytometry system of claim 3, wherein the waveform analysis device is configured to:
apply a first threshold voltage;
analyze the digitized waveform data using the first threshold voltage;
apply a second threshold voltage different than the first threshold voltage; and
analyze the digitized waveform data using the second threshold voltage without re-running the particles through the interrogation location.

7. The flow cytometry system of claim 3, wherein the waveform analysis device is configured to:
apply first and second thresholds to the digitized waveform data; and
generate event data including a sequence of digital values from the digitized voltage waveforms by ensuring each digital value in the sequence of digital values is greater than the first threshold and less than the second threshold without re-running the particles through the interrogation location.

8. The flow cytometry system of claim 3, wherein the waveform analysis device is configured to automatically determine the one or more adjustable threshold voltages for individual pulses of the digitized waveform data.

9. The flow cytometry system of claim 3, wherein the waveform analysis device applies the one or more adjustable threshold voltages after the digitized waveform data is acquired from the waveform acquisition device.

10. The flow cytometry system of claim 1, wherein the graphics processing unit is configured to process the event data to compute skew and kurtosis for each of the particles.

11. The flow cytometry system of claim 1, wherein the graphics processing unit is configured to apply a fast Fourier transform (FFT) to the digitized waveform data to generate vector valued data for each of the particles.

12. The flow cytometry system of claim 1, wherein at least one adjustable threshold voltage of the one or more adjustable threshold voltages is a non-constant value.

13. The flow cytometry system of claim 1, wherein the waveform acquisition device is configured to continuously digitize the analog waveform data including time between the particles when a particle is not interrogated by the laser.

14. The flow cytometry system of claim 1, wherein the waveform acquisition device is configured to continuously digitize the analog waveform data without using a threshold voltage.

15. The flow cytometry system of claim 1, further comprising:
a sheath pressure sensor configured to detect a fluid pressure; and
a laser sensor configured to detect a light intensity of the laser;
wherein the waveform acquisition device is configured to continuously digitize the fluid pressure and the light intensity.

16. A method of analyzing particles flowing through a flow cytometer, the method comprising:
directing a fluid stream of particles through an interrogation location;
emitting light toward the interrogation location to produce light signals from the particles;
converting the light signals to analog waveform data;
continuously digitizing the analog waveform data including time between the particles when a particle is not interrogated by the laser; and
applying one or more adjustable threshold voltages to the digitized waveform data to extract event data from the particles.

17-20. (canceled)

21. A method of post-processing flow cytometry data, the method comprising:
accessing digitized waveform data from a computer readable storage device after interrogation of particles in a sample has been completed by a flow cytometer;
determining a threshold voltage;
applying the threshold voltage to the digitized waveform data; and
characterizing the particles in the sample after applying the threshold voltage.

22. The method of claim 21, wherein the threshold voltage is specified by a user input.

23. The method of claim 21, further comprising:
after characterizing the particles, determining a second threshold voltage different than the threshold voltage previously applied to the digitized waveform data;
applying the second threshold voltage to the digitized waveform data; and
recharacterizing the particles in the sample after applying the second threshold voltage.

24. The method of claim 23, wherein applying the second threshold voltage to the digitized waveform data is performed without re-running the sample through the flow cytometer.

25-30. (canceled)
